(19) United States
(12) Patent Application Publication
(10) Pub. No.: US 2025/0143878 A1
(43) Pub. Date: May 8, 2025

(54) TRANSCATHETER VALVE INTRODUCER SYSTEM AND METHODS

(71) Applicant: Foldax, Inc., Salt Lake City, UT (US)

(72) Inventors: Garrett Johnson, Costa Mesa, CA (US); James Wittell, Salt Lake City, UT (US); Megan Nguyen, Salt Lake City, UT (US); Praveen De Silva, Irvine, CA (US); Marek Lhotak, Irvine, CA (US)

(21) Appl. No.: 18/835,852

(22) PCT Filed: Feb. 27, 2023
(86) PCT No.: PCT/US2023/013994

§ 371 Date: Aug. 5, 2024

Related U.S. Application Data
(60) Provisional application No. 63/314,344, filed on Feb. 25, 2022.

(51) Int. Cl. A61F 2/24

(52) U.S. Cl. CPC A61F 2/2436; A61F 2/2418; A61F 2220/0025

(57) ABSTRACT

A delivery device is described for use in a TAVR procedure for replacement of a diseased cardiac valve with a prosthetic valve using a minimally invasive procedure. The invention includes structural features of a prosthetic valve that are mated to corresponding structural features on a catheter-based delivery system. Individual structures and operations of the delivery system are specially designed for the TAVR procedure and operate to improve the delivery and placement of the prosthetic valve at the native annulus such that the surgeon can orient and release the prosthetic valve in a controlled manner to improve the placement and deployment of the prosthetic valve.

### BACKGROUND OF THE INVENTION

Transcatheter aortic valve replacement (TAVR) is rapidly becoming an established treatment for replacing a diseased heart valve. TAVR is a less invasive alternative to traditional surgical aortic valve replacement and offers equivalent or superior outcomes for many patients. In the TAVR procedure, rather than a large open surgical incision, and the necessary use of a heart lung machine, a prosthetic valve is placed inside a thin catheter that is introduced to the patient through a small incision near a patient's artery, typically the femoral artery in the upper leg, and advanced to the diseased valve location inside the heart where the prosthetic valve is deployed by releasing the new prosthetic valve from the end of the catheter to replace the existing, diseased heart valve without any large incision and without stopping the heart. The use of TAVR has increased rapidly in patients of all risk categories and now comprises at least 12.5% of all aortic valve replacements. The success and increase in use of the TAVR procedure are a result of advances in valve design technology and surgical instrumentation developed specifically for the TAVR procedure, greater operator experience, and a record of improved patient outcomes where the TAVR procedure is used rather than open surgery.

TAVR has been a rapidly evolving field since the first valve was implanted in a patient with inoperable heart valve disease, termed “severe aortic stenosis” in 2002, amidst strong early criticism of the TAVR procedure. TAVR has now been performed in over 400,000 patients worldwide. Randomized controlled clinical trials have demonstrated the safety and efficacy of the TAVR procedure, first in patients where an open surgical approach was not possible, and then in high-risk, intermediate, and most recently some low-risk patients rather than the open surgical procedure. However, even more widespread acceptance of TAVR requires overcoming many existing technical challenges, especially the need to demonstrate long-term viability of implanted valves using the TAVR procedure, as well as the continuing design of prosthetic valves specifically designed for TAVR and the functionality of delivery systems used in the TAVR procedure.

Progressive heart valve disease is common in developed countries and is strongly associated with age, life style, and other forms of vascular disease. As the population ages, progressive and irreversible heart valve disease will become an increasingly significant health burden and the number of valve replacement procedures is expected to significantly increase. One particular type of valve disease, aortic stenosis, increases with age and affects 2.8% of patients aged 60-74 years and 13.1% in patients 75 years and older, which corresponds to approximately 16.1 million people. The estimated number of patients with severe aortic stenosis is 3.2 million, and approximately one million of them are eligible for TAVR. TAVR is expected to be an increasingly important option for minimally invasive valve replacement across a variety of different types of valve disease.

For example, one recent trial showed that TAVR was superior to more invasive surgical procedures for the primary endpoints of all-cause mortality, stroke, rehospitalization, and new-onset atrial fibrillation at one year given these results. Based on these results, the United States Food and Drug Administration (FDA) expanded the indications for TAVR to a broader patient population. Two-year follow-up data in low-risk TAVR patients showed persistent superiority for the combined primary endpoint (death, stroke, or cardiovascular rehospitalization) and rehospitalization alone and persisted at 1 year with some diminishment in superiority of the TAVR procedure at two years. Therefore, despite more widespread adoption, the adoption of the TAVR procedure would benefit from the development of new valves and new delivery systems that render the procedure more effective, reliable, reproducible, and able to demonstrate long-term advantages crossed the broadest possible target patient population.

The TAVR procedure requires a specially designed prosthetic valve that can be collapsed such that the overall diameter is small enough to be inserted at the end of the catheter. As noted above, the catheter is introduced into the patient's vasculature and advanced to the point of the diseased heart valve where the prosthetic valve is deployed to replace the existing valve while the patient's heart continues to beat. For this replacement to be successfully achieved, the prosthetic valve must expand into the space occupied by the native valve, be secured to the annulus that surrounds the existing valve, and take over the function of the existing valve by replacing the structure of the existing valve that remains in place even after the prosthetic valve is deployed. Thus, the prosthetic valve must deploy from the end of the catheter in a series of structural configurations that is controlled and precise so that the location and orientation of the new prosthetic valve at the native valve is stable and reliable. Once the surgeon determines that the location of the prosthetic valve is correct, and that the new prosthetic valve is functioning in place of the diseased valve, the catheter is withdrawn leaving the prosthetic valve in place. Thus, the TAVR procedure requires both special instrumentation for introduction of the valve through a minimally invasive process, as well as a specially designed prosthetic valve.

During the initial development and long-term expansion of the use of the TAVR procedure, a number of different designs for the prosthetic valve have been used and a number of different delivery systems have been used. Currently, in consultation with heart surgeons, several researchers and engineers are investing intensive efforts to develop both new prosthetic valve designs and configurations of new delivery systems to encourage wide adoption of the TAVR procedure in the increasing patient population. Because of the differing designs of prosthetic valves capable of being used in the TAVR procedure, introducer/delivery systems also have to be designed to match the delivery parameters of the specific prosthetic valve used with a particular patient. Ideally, a cooperation between the specific design of the prosthetic valve and the transcatheter delivery system will enable superior outcomes and increase the adoption and applicability of the TAVR procedure.

Therefore, although the TAVR procedure offers great promise and adoption continues to expand, there is a need for improved valve designs and delivery systems specially matched to the unique prosthetic valves used in the TAVR procedure.

### SUMMARY OF THE INVENTION

The current invention is a transcatheter valve delivery system that has structural features to improve the TAVR procedure for a matched prosthetic valve design and has several design features that enable the precise placement and deployment of the prosthetic valve specially designed for the TAVR procedure. The delivery system contains structural features that improve multiple different aspects of the TAVR procedure, including loading of the valve at the distal end of the catheter by reducing the diameter of the prosthetic valve from an expanded to a collapsed condition. Also, features of the delivery system include structures that promote preferable folding and orientation of the valve leaflets when the prosthetic valve is reduced in diameter from the expanded configuration to the collapsed configuration as the valve is introduced into the distal end of the catheter. Aspects of the invention include novel structures, cooperation between novel structures and traditional catheter structures and novel methods for manipulating and orienting the valve relative to the foregoing.

Additional features help to ensure that the prosthetic valve, when maintained in a collapsed configuration, is more accurately and precisely deployed and is not damaged in the process of being placed into the collapsed configuration at the distal end of the catheter. Other aspects of the delivery system relate to the proper orientation and placement of the valve as the catheter is advanced through the vasculature of the patient from the minimally invasive incision to the point at which the valve is deployed by manipulation of controls integral to the delivery system. These include both fixtures retaining the valve in the collapsed configuration as well as structural designs of the catheter itself along the length thereof to promote atraumatic placement through the vasculature to the native annulus. Finally, certain features of the delivery system are designed to ease manipulation of the catheter and the valve deployment structures by the surgeon using design features in the structure and function of the handle of the delivery system using controls that are hand-manipulated by the surgeon.

In some embodiments, the invention is comprised of a combination of modifications to the delivery system including at least certain specific modifications to the distal portion of the catheter and associated structures that maintain the valve in a preferred configuration together with features of the catheter itself. To be collapsed into a significantly reduced diameter, the prosthetic valves suitable for the TAVR procedure have specialized design features at both the inflow and outflow portion of the prosthetic valve, together with a collapsible leaflet structure, so that the diameter of the valve is reduced while the length of the valve increases along a linear axis thereof. The leaflets of the prosthetic valve must fold internally to the catheter along a length thereof during the introduction of prosthetic valve into the distal end of the catheter and during the time when the valve is compressed therein.

In addition to structural design features that enable placement of the valve in a compressed configuration, and selective deployment of the valve in the native annulus, the catheter may include dedicated or separate structures for recapture of the valve after deployment. Although relatively rare, in some circumstances the deployment of the valve from the distal end of the catheter results in an imperfect placement of the valve in the native annulus. Under such circumstances, the surgeon may wish to recapture the valve into the distal end of the catheter for repositioning or removal. Recapture is permitted by cooperating structural features between all of the control mechanisms for catheter, structural features on the prosthetic valve, and a dedicated recapture structural design configuration located at the distal end of the catheter. The key feature of the recapture mechanism is the ability to return the prosthetic valve from a partially or totally expanded configuration and to retain a re-collapsed valve in the distal end of the catheter once the recapture decision has been implemented by the surgeon.

A combined structure located at the most distal end of the catheter is a nosecone having an internal diameter and an external diameter with a tip at the distal most point thereof and an inner channel comprised of a guidewire lumen and a guidewire disposed within a specified portion of the lumen. In some embodiments, the design of the nosecone is modified relative to the guidewire lumen that traverses the length of the catheter and has an improved shape and dimension to support the prosthetic valve in the collapsed state. Specifically, the configuration of the structure of the nosecone relative to the guidewire lumen enables a smaller diameter atraumatic tip at the distal most end of the nosecone. Furthermore, a mechanical interface may be introduced to the body of the nosecone, or the nosecone may be over molded thereabouts, to provide an improved structural bonding between the nosecone and the guidewire lumen and preferred overall dimensions of the nosecone portion. A proximal portion of the nosecone can be expanded in volume and dimensions and extended proximally and tailored to meet with the most distal portion of the prosthetic valve. The proximal portion of the nosecone is stepped down to align with the inner diameter the capsule 25. The outer diameter of the nosecone, just distal of the stepped down portion, is the same outer diameter as the capsule 25 to facilitate a smooth transition. The extended axial portion of the nosecone is designed and configured that the prosthetic valve has room in the capsule 25 to reassume a reduced diameter during a recapture procedure if necessary. Under such circumstances, the proximal nosecone would extend into the inner diameter of the prosthetic valve and assist with reducing the overall inner and outer diameter of the prosthetic valve during recapture. the proximal portion of the nosecone is stepped down to align with the ID of the capsule 25. The OD of the nosecone, just distal of the stepped down portion, is the same OD as the capsule 25 to facilitate a smooth transition. The axial position of the nosecone is set and the dimensions pre-determined such that the valve has adequate clearance in the capsule 25 to facilitate crimping and recapture if needed.

In combination with either a conventional or modified design for the nosecone, an inner ring valve support structure surrounds the guidewire and that maintains the prosthetic valve in a preferred configuration in the collapsed state. The inner valve support structure has a recessed portion formed as a negative or mirror image of at least a portion of the prosthetic valve in the collapsed state, such that the valve in the collapsed state is maintained in the form and orientation of the frame support structure when introduced into the distal end of the catheter. In the collapsed configuration, the outer diameter of the inner ring valve support containing the prosthetic valve is substantially similar to the native diameter of the entering valve support structure because the portion of the prosthetic valve designed to fit within the negative or mirror image of the inner valve support structure is contained substantially therein. In this configuration, the portion of the prosthetic valve engaged by the inner ring valve support structure can be incorporated into the distal end of the catheter and maintained in a preferred configuration and a constant diameter defined by the inner ring valve support structure. Each of these features improves the configuration of the valve when maintained at the distal most portion of the catheter and facilitates both deployment and avoids damage to the prosthetic valve, including the folded leaflets, that may occur while the valve is loaded into the distal end of the catheter and maintained in the collapsed state.

Another advantage is the ability to radially orient the inner ring valve support structure to control the deployment of the prosthetic valve to ensure that the commissures are oriented properly with respect to the native valve commissures. A distal portion of the inner ring valve support structure may have a releasable retention feature such as a post or configuration of the distal end of the channel forming the negative or mirror image that engages a proximally located corresponding structure on the prosthetic valve to retain the valve in the support structure and to maintain the valve in a predetermined configuration when disposed in the collapsed configuration within the capsule 25 of the catheter and to enable controlled release of the prosthetic valve from the inner ring valve support structure.

In addition to the modified structures at the distal end of the catheter, the delivery system also includes a kit having a distal loading funnel for reducing the diameter of the prosthetic valve along its length for preparation for insertion into the distal end of the catheter. As explained in more detail below, the process of loading the prosthetic valve into the distal end of the catheter requires reducing the diameter of the valve while loading the valve into the distal end such that the first expanded diameter is reduced to the second collapsed diameter while maintaining the valve leaflets, commissure support structures and other features of the prosthetic valve in a determined orientation for complete placement of all portions of the prosthetic valve, such that the stent portion, the valve portion, and the crown portion, are aligned for introduction into the distal portion of the catheter. Therefore, the distal loading funnel may comprise a separate part of an assembly or kit used to reduce the prosthetic valve from the completely expanded to at least a partially compressed state in the catheter and may be complementary to other features on the distal portion of the catheter that facilitate placing the valve in the collapsed configuration.

Once placed in this configuration, together with a funnel structure located at the distal end of the catheter and proximal to a surface at the distal end thereof to facilitate final reduction of the diameter of the prosthetic valve into the final compressed configuration inside the catheter in the configuration as introduced to the patient vasculature. This funnel is formed in the distal-most surface of the catheter or as a component of a loading kit.

The catheter used to introduce the prosthetic valve by traversing human vasculature must be highly bendable along a length thereof while also having the capability to assume a three dimensional configuration that conforms to the human vascular pathway for advancement of the catheter from the entry point to the diseased heart once introduced into the vasculature of the patient and the prosthetic valve advanced to the native annulus. For example, where the catheter is introduced through the inner thigh of a patient at the femoral artery, the catheter will have a lengthy portion that is roughly linear until traversing the descending thoracic aorta and approaching the aortic arch where the catheter must typically the steered to bring the distal end, containing the prosthetic valve, and to the location of the diseased valve. A substantial portion of the entire length of the catheter may be laser cut so that the portion that enters the femoral artery through the portion that deploys the prosthetic valve has a specifically designed range of flexibility and tensile/column strength so that the catheter is pliable enough to orient the prosthetic valve at the native annulus, but rigid enough to be advanced through the patient vasculature. Thus, the portion of the catheter that traverses the aorta typically must conform to a curve that has a deflection angle greater than 90°.

Because the general shape of the catheter when introduced into the vasculature of the patient and advanced to the point of deployment of the prosthetic valve can be predicted based on standard human physiology. Knowing this configuration, the catheter can be designed to have the capability to assume a predetermined curvature conforming to the length and the three dimensional configuration can be engineered into the structure of the catheter by adapting selected sections along the length thereof to assume a predetermined curve. Although a number of catheter designs are known to allow a surgeon to steer a catheter to some extent, the present invention provides a catheter that is structurally designed to conform to the curvature of a human vasculature by virtue of individual segments or splines along the length of the catheter that are structurally formed and oriented to introduce a predetermined curvature the aorta and places the prosthetic valve proximate to the point of deployment at the native annulus.

As will be apparent from this description, the entire procedure wherein the catheter is guided to the diseased valve, the prosthetic valve is deployed, and the delivery system withdrawn from the patient is achieved by manual manipulation of controls at the proximal end of the catheter. The invention includes a specially designed handle system having internal mounted rails for steering of the catheter and deployment of the prosthetic valve. The structure of the handle and design of the controls allows the surgeon to readily advance the prosthetic valve through the patient's vasculature, manipulate the configuration of the catheter to bring the prosthetic valve in conforming contact with the diseased valve, and to accurately deploy the prosthetic valve.

The delivery system of the invention also includes a separate structure for preventing emboli from entering the patient's circulation during placement of the prosthetic valve. Due to the presence of calcification, loose tissue, and disease surrounding a failing native cardiac valve, the process of placing the new prosthetic valve can dislodge small particles formed of tissue, cellular debris, scar tissue, calcified particles and other extraneous matter that can be harmful when dislodged to enter a patient's circulation. In one optional embodiment of the invention, an embolic protection device located proximally of the structure at the distal end of the catheter containing the compressed configuration valve that is deployed in the TAVR procedure. The invention also includes the specific method steps of deploying the embolic protection feature coincident with causing the catheter to traverse a portion of the vasculature and with deployment of the prosthetic valve.

The invention also includes assembled systems, kits, and methods for using the individual components and structures of the delivery system herein including particularly methods for combining features of a selected prosthetic valve with the features of this delivery system for use in combination and a TAVR procedure as described herein. The invention also includes literature, instructions for use (IFUs) and companion structures, compounds, solutions and other instruments for preparing the prosthetic valve and delivery system for use with a particular patient. The methodology also includes imaging apparatus and companion surgical equipment that facilitates the introduction, placement, and deployment of the prosthetic valve using the delivery system as described herein. From this description and the accompanying Figures, other systems, devices, methods, features and advantages of the subject matter described herein will be apparent to those with skill in the art of transcatheter valve replacement.

### DESCRIPTION OF THE DRAWINGS

The details of the subject matter of the present invention, both as to structure and operation are apparent from study of the accompanying figures in which reference numerals generally refer to similar parts. The components in the figures are not necessarily to scale, except where as indicated by quantitative measurements of the size, scale, and relative orientation of various structures. The figures convey inventive contents, where relative sizes, shapes, and other detailed attributes may be illustrated schematically rather than literally or precisely.

FIG. 1A shows a traditional nosecone typical in the prior art having a tapered outer surface extending from a proximal portion to a distal tip and an internal guidewire lumen traversed by a guidewire. FIG. 1B is a modified nosecone having an interior guidewire lumen offset that terminates proximally from the distal tip and may have an internal mechanical interface proximate to the distal end of the guidewire lumen to increase bond strength of the nosecone to the guidewire lumen. The body of the nosecone has an extended proximal portion designed to support a valve to prevent undesirable enfolding of valve leaflets. FIG. 1C is a compressible support located just proximal of the most proximal portion of the nosecone and disposed within the distal most portion of the prosthetic valve.

FIG. 2A is an inner ring valve support member that is traversed by the guidewire at an interior portion and features a cavity shaped as a negative or mirror shape of the most proximal portion of the prosthetic valve and an optional attachment feature for attachment to the inner ring valve near a distal most portion of the prosthetic valve. FIG. 2B shows the orientation of the valve contained within the negative or mirror shape to illustrate the orientation of the inner ring valve support and the proximal end of the valve in the compressed configuration just prior to being loaded in the catheter.

FIG. 3A shows a loading kit assembly having both a separate distal loading funnel and a funnel-shaped portion formed in the distal most portion of the catheter to facilitate a smooth transition of the prosthetic valve from the expanded or partially expanded state to the collapsed configuration inside the catheter during the valve loading procedure. FIG. 3B is the combination of the nosecone, the distal loading funnel containing a portion of the prosthetic valve, the inner ring support member and the distal portion of the catheter traversed by the guidewire and separated in space to show the mating relationship of each prior to the valve loading procedure. A distal loading funnel oriented proximate to the end of the catheter that is part of the loading kit and maintains the prosthetic valve in a partially compressed state such that the proximal end of the valve is shaped to be introduced to the distal end of the catheter. FIG. 3C shows the proximal portions of the valve disposed in the inner ring valve support member just prior to introduction to the distal end of the catheter. FIG. 3D shows the internal and external views of the loading kit showing the distal loading funnel in mating engagement with the most distal portion of the catheter and showing the positioning of the nosecone described in FIG. 1B. FIG. 3E shows the configuration of paired inner loading cones within the outer loading cone of the loading kit. The positioning of an inner loading cone locking ring and a separate inner loading cone positioned distally of the loading funnel.

FIGS. 4A and 4B are one graphic concept of a recapture system having a deployable and rotatable pair of recapture extensions that lie internal and substantially parallel to the capsule 25 at the distal portion of the catheter. FIG. 4A shows the recapture extensions funnel disposed within the outer diameter of capsule 25 prior to deployment. FIG. 4B shows a recapture funnel extending beyond the most distal portion of the capsule 25 and an expanded state and prepared to receive the proximal portion of the prosthetic valve to draw the valve back into the catheter if recapture after partial or complete deployment is required. FIG. 4 C shows an alternate embodiment where the recapture extension is a sliding sleeve that extends from the distal end of the catheter to recapture the prosthetic valve.

FIG. 5A is a linear configuration of an elongated catheter element of the delivery system having laser cut spines that enable the operator to flex the length of the catheter, and particularly the distal end of the delivery system, in a desired direction and a magnified section to show how the shape of the individual spines are manufactured to facilitate the ability to change configuration along a linear axis thereof. FIG. 5B shows a sectional view of the distal end of the catheter showing the laser cut hypotube forming the capsule 25 and a locking ring 39 (see FIG. 3E) proximal to the capsule 25 the orientation of a flex layer hypo one, a pull wire, and outer layer of the catheter comprising a transition ring and an outer layer hypotube. The spine of the capsule 25 is keyed with the spine of the inner ring layer and is also hydrophilically coated to allow ease for insertion, and crossing the aortic arch. FIGS. 5C and 5D show alternate details of the laser cut spines of the catheter that enable flexibility along the length thereof while maintaining structural stability. FIG. 5E shows the delivery system wherein the catheter is deployed in the vasculature of a patient and wherein the rotational and flexible dimensions may be predetermined and oriented in line with the anatomical orientation of the abdominal artery, descending thoracic aorta, aortic arch, and descending aorta to bring the prosthetic valve to deployment in the heart.

FIG. 6 is the overall configuration of the delivery system showing multiple individual components as described in the other Figures and as described herein showing the proximal most handle portion manipulated by the surgeon, the elongated catheter and valve delivery structures located at the distal most portion of the delivery system.

FIG. 7A is an internal view of a rail mounted delivery system having steering and deployment knobs accessible around the outer surface thereof that are manipulated by the surgeon during the TAVR procedure. FIG. 7B is an internal view of the rail mounted delivery system showing the operation of the outer layer hypo to the flex layer hypo to and outer layer and flex layer barrels.

FIG. 8 is an internal configuration of the rail mounted delivery system showing the internal configuration of the stationary layer, stationary layer handle adapter, capsule handle adapter, inner ring handle adapter, and flex handle adapter oriented relative to the mounting rails.

FIG. 9 is an embolic protection mechanism that may be oriented and deployable in an annular configuration around the catheter that can be deployed during a TAVR procedure between the prosthetic valve and the downstream blood flow of the vasculature of the patient, preferably located just proximal of the delivery system capsule. Particularly during the deployment of the prosthetic valve at the native annulus, and during introduction and advancement of the catheter the risk of emboli can be mitigated. The embolic protection mechanism may be located proximate to the capsular portion containing the prosthetic valve or may be deployed separately.

FIG. 10 is a delivery system having an outer shaft, and preferably with a fixed orientation to the commissures of the prosthetic valve fixed at the distal end such that manipulation of using a control knob at the handle of the delivery system allows the surgeon to selectively position the prosthetic valve structures in a rotational fashion relative to the axial length of the catheter and the native annulus and allows the surgeon to selectively position the prosthetic valve relative to existing native structures, such as to accommodate features of and individual patient anatomy or to avoid coronary arteries.

FIG. 11A is an exemplary embodiment of a prosthetic valve for use in a TAVR procedure. The valve has a skirt feature, a frame for commas your support structures in a stent embodiment and having a superior halo feature at the outflow section comprised of a single continuous wire terminating in proximal attachment features. FIGS. 11B and 11C are detailed views of a rivet feature incorporated into the frame of the prosthetic valve between the body of the frame and a superior frame structure that forms a halo at the downstream end of the prosthetic valve.

### DETAILED DESCRIPTION OF THE INVENTION

Before the present subject matter is described in detail, it is to be understood that this disclosure is not limited to the particular embodiments described and may vary. It is also to be understood that the terminology used herein is for the purpose of describing particular embodiments only, and is not intended to be limiting, since the scope of the present disclosure will be limited only by the appended claims.

The example embodiments described herein relate to improved implantable prosthetic valves, such as prosthetic heart valves having a support structure, stent, or frame coupled with two or more leaflets, and techniques for the manufacture of implantable valves. These embodiments are particularly suited for artificial polymeric leaflets, and the resulting artificial valves offer advantages comparable to current approaches with the added benefit of a longer life span. Valves with polymer-based leaflets are advantageous because polymers can offer the same structural support as biological tissue, while being much thinner and allowing the valve to be more easily collapsed for delivery. This in turn results in less stress on the polymer as it is compressed or contracted which prevents long-term degradation of the valve leaflets. In addition, the manufacturing methods described herein permit fabrication of a valve without suturing leaflets to a support structure or stent, thus promoting high quality repeatable results. In some circumstances, the structures and systems described herein are uniquely suited for polymer-based leaflets while others are broadly applicable to both polymer-based and tissue-based prosthetic valve designs. Examples of polymeric materials for the prosthetic valves described herein are provided in the following U.S. Pat. Nos. 10,266,657; 10,723,844; and 11,053,542 that are specifically incorporated by reference herein.

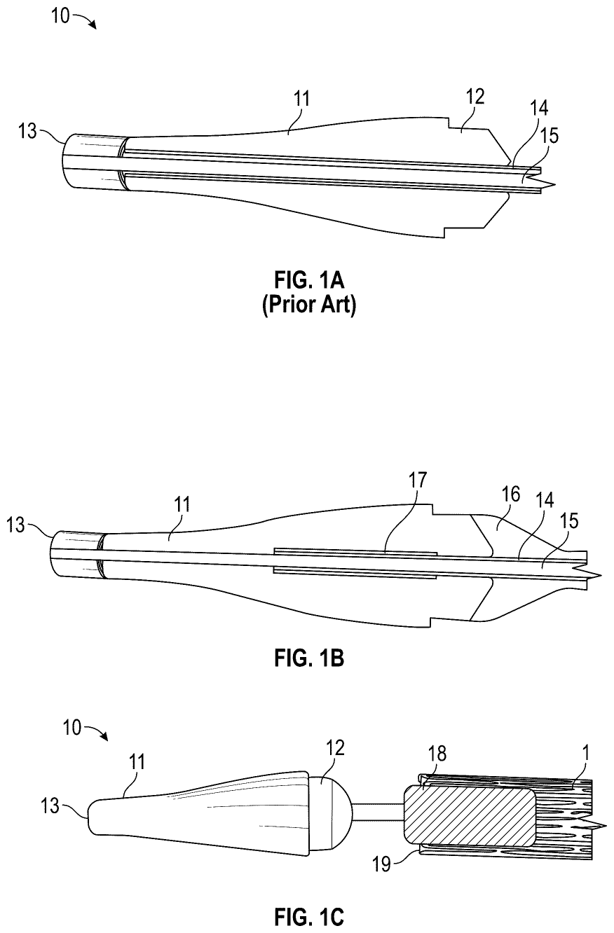

FIG. 1A shows a traditional nosecone 10 typical in the prior art having a tapered outer surface 11 extending from a proximal portion 12 to a distal tip 13 and an internal guidewire lumen 14 traversed by a guidewire 15. FIG. 1B is a modified nosecone designed for a TAVR procedure having an interior guidewire lumen offset 17 that terminates at a predetermined distance proximally from the distal tip 13 and may have an internal mechanical interface 18 that is bound or otherwise molded to the guidewire 15 proximate to the distal end of the guidewire lumen 15 to increase bond strength of the nosecone 10 to the guidewire lumen 15. The body of the nosecone has an extended proximal portion 16 designed to conform to the inner diameter of a prosthetic valve, particularly the most distal portion thereof when the valve is loaded into a delivery catheter, and to support the valve during placement within the interior space at the distal end of the catheter 19 and to prevent undesirable orientation of the valve leaflets and frame support structures when the prosthetic valve is reduced to the contracted or collapsed configuration. By extending the nosecone proximally, the proximal extension 16 acts as a support structure acting as a platform or shelf to occupy the space where undesirable mis-folding of leaflets could occur. Misalignment of the valve frame and support structures within the catheter or during loading can lead to asymmetric crimping or mispositioning of the prosthetic valve. For example, the support outer diameter (OD) cannot be too small where the structure would not offer enough support to the valve structure along the length thereof, but also must not be too large because the valve would be prevented from fitting into the delivery structure such as a capsule or other structure that maintains the valve in the collapsed state at the distal end of the delivery catheter.

The nosecone 10 can be fabricated by traditional techniques using known biocompatible, non-immunologically reactive materials including injection molding from Pebax, 3D printed from silicone, molded from PTFE, and other known techniques. During fabrication, imaging materials such as barium sulfate and other known imaging compounds can be included in the biocompatible material to enable viewing of the nosecone under fluoroscopy or other conventional medical imaging techniques. The body of the nosecone 10 is radiopaque and can be visualized under fluoroscopy during the TAVR procedure.

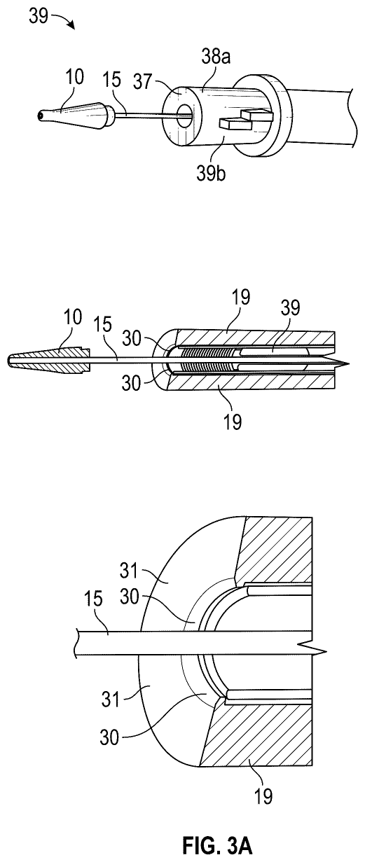

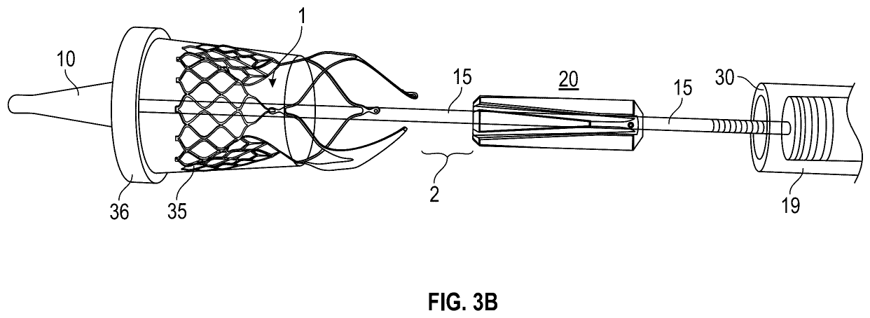

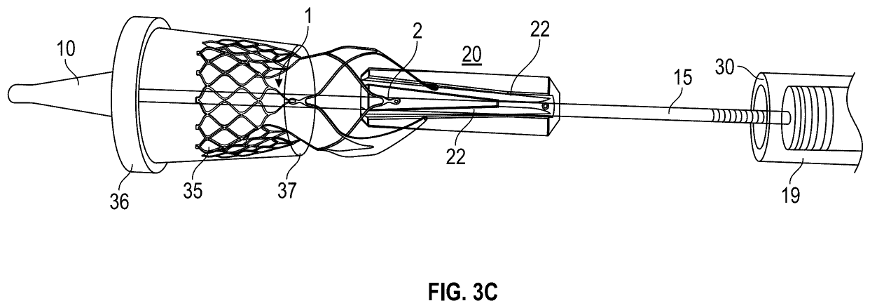

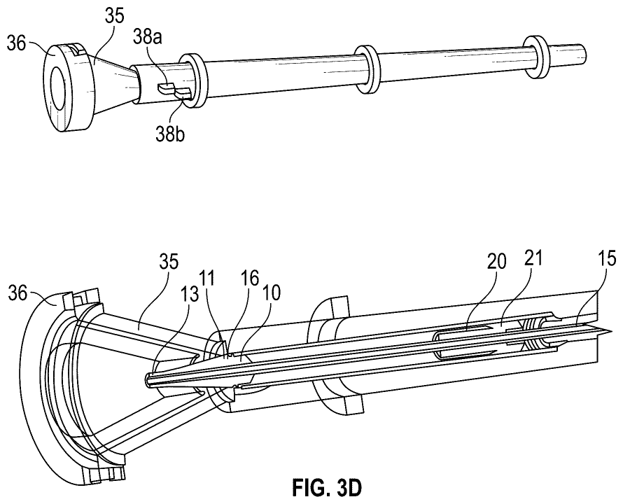

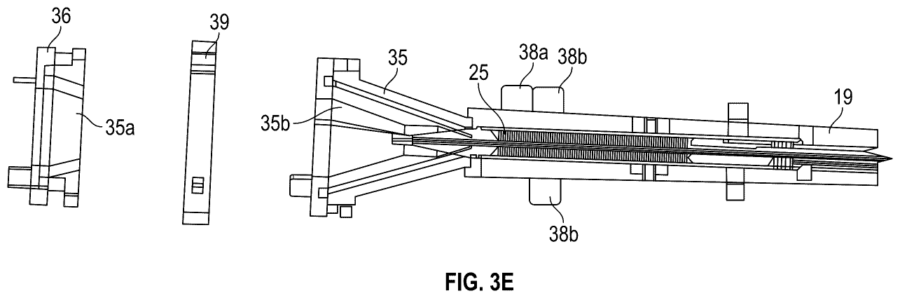

In one embodiment, a maximum outer diameter of the nosecone is approximately 20 French that tapers down to a reduced outer diameter of approximately 18 French for the capsule 25 contained in the interior space at the distal end of the catheter 19 (see FIG. 3A below); the prosthetic valve conforms to the outer portion of the distal extension 16 of the nosecone 10 when the prosthetic valve is loaded into the catheter and progresses from the expanded to the compressed configuration as described in more detail below.

The location of the guidewire lumen is offset 17 at a point proximal to the distal tip 13 and at a position that is intermediate within the body of the guidewire 15 can shorten the length of the guidewire lumen 14 enabling the creation of an atraumatic distal tip 13 having a smaller diameter D1 compared to the traditional diameter of a distal tip. FIG. 1C is a compressible support 18 located just proximal of the most proximal portion 12 of the nosecone 10 and disposed within the distal most portion of the prosthetic valve 1 to maintain the valve in a fixed configuration inside the distal end of the catheter 19. In use, the proximal most portion of the nosecone 12 engages the compressible portion 18 that itself surrounds and is traversed by the guidewire 15 while the catheter 19 traverses the vasculature of the patient during the TAVR procedure.

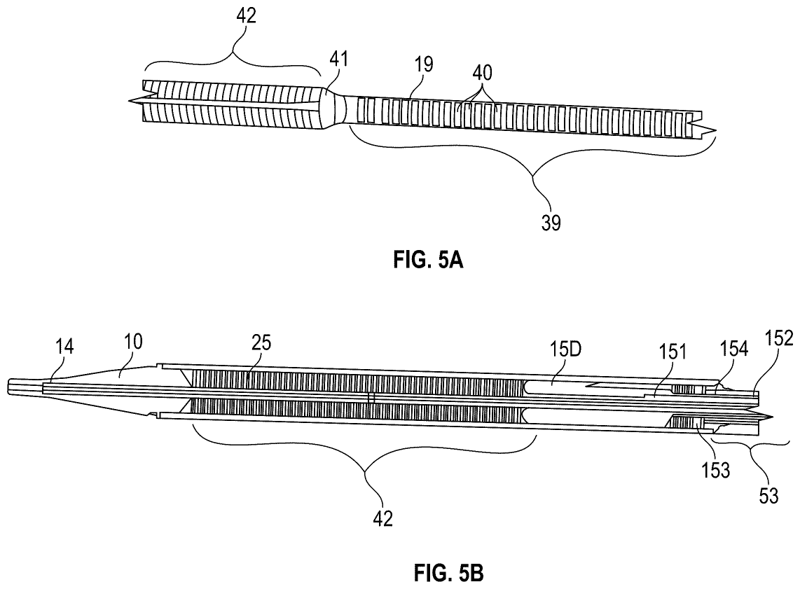

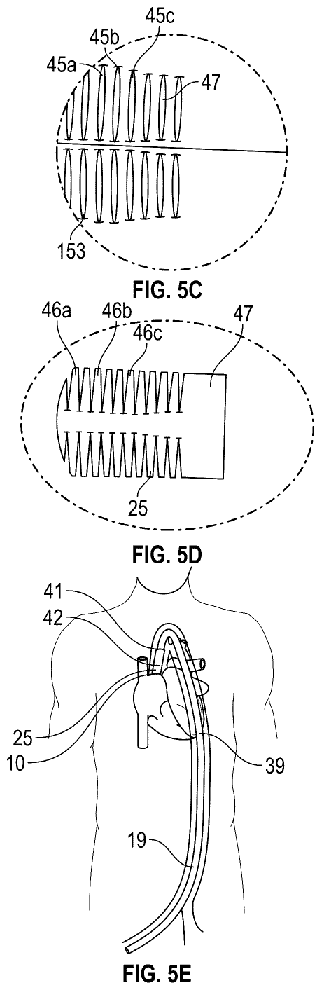

A smaller diameter, more atraumatic tip 13 of the nosecone 10 is desirable to enable easier femoral access and to lower the risk of vascular access complications as the tip of the catheter is advanced through the vasculature of the patient. See FIG. 5B. By embedding the guidewire lumen 14 at an offset position set back (proximally along the guidewire 15) from the nosecone tip 13, a nosecone tip 13 having a smaller diameter can be formed from conventional materials and without altering the traditional use of the nosecone device 10 in the traditional context of a transvascular procedure. In combination with the existing system, the alteration in the dimensions of the nosecone tip 13 enhances atraumatic delivery in dimensional orientation of the prosthetic valve.

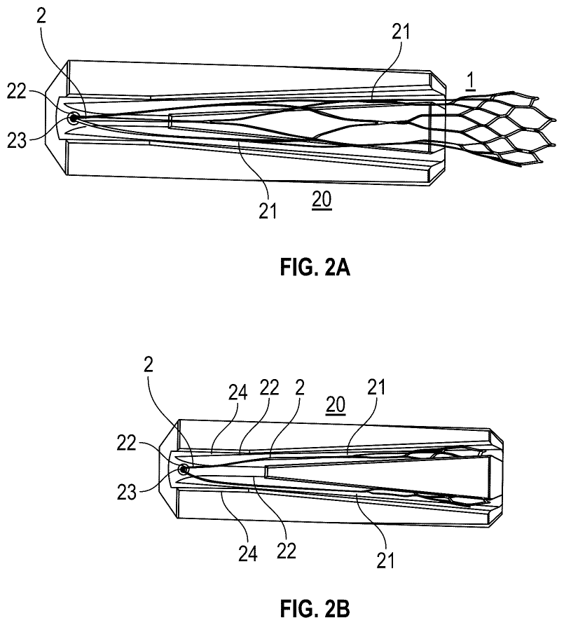

FIG. 2A is a segment of an inner valve support 20 that supports the frame of the prosthetic valve 1 in the collapsed configuration as mounted on the catheter 19. The inner valve support 20 is traversed by, and circumferentially disposed about, the guidewire lumen along the entire length of the interior portion thereof and features a cavity 21 shaped as a negative or mirror shape of the most proximal portion 2 comprising the halo or outflow aspect of the prosthetic valve 1 and an optional attachment configuration 22 for maintaining engagement of the inner valve support 20 near a distal most portion of the prosthetic valve 1. In a prosthetic valve 1 featuring three leaflets and three respective commissure support structures, a trio of the inner valve support 20 fragments would be arranged in an annular fashion surrounding the guidewire lumen 15 so that each of the respective proximal portions 2 comprising the halo portion of the prosthetic valve 1 would each fit within a single cavity 21 and all three of the cavities 21 would be disposed in an annular orientation around the guidewire lumen 15 and each of the cavities 21 would contain or be engaged by the three respective distal portions 2 of the prosthetic valve 1. The attachment configuration 22 maintains the proximal portions to of the prosthetic valve 1 securely engaged or contained within the cavity or cavities 21.

As shown in FIG. 2A, the attachment configuration 22 may comprise a dedicated fixture such as a post 23 positioned at the most proximal portion of the cavity 21. The attachment configuration 22 may also be comprised of the combination of the shape of the distal most portion of the cavity 21 and distal most portion of the valve 21, including where the halo of the valve is compressed to fit in mating engagement with the interior portion of the cavity 21. In the embodiment of FIG. 2B, a portion of the cavity 21 at the more distal and has a broader width or depth than a more proximal portion to form an hourglass shape 24 that retains the position of the distal most portion 2 of the valve against movement in an axial direction along the length of the cavity 21 and assists in maintaining the length of the structural portion of the prosthetic valve 1 in the cavity or recesses of the inner valve support 20. FIG. 2B also shows the orientation of the proximal portion 2 of the prosthetic valve 1 contained within the negative or mirror shape cavity 21 to illustrate the combined orientation of the inner valve support 20 and the proximal end 2 of the prosthetic valve 1 in the compressed configuration and while the main body of the prosthetic valve 1 is still in an expanded configuration just prior to being loaded into the distal end of the catheter 19.

The inner valve support 20 creates higher column strength in the compressed state of the valve which enables the prosthetic valve 1 to be delivered from the catheter 19 while maintaining a configuration and orientation to enable the prosthetic valve 1 to be released from the catheter and deploy at the native annulus under control of the surgeon. The cavity 21 ideally extends a couple of millimeters proximal of the halo of the valve 1 to fit the locking ring connector piece. The ID of the capsule 25 and the OD of the locking ring are held in close conformity, for example less than 0.005″ combined. A larger gap risks displacement of the prosthetic valve 1 during loading or deployment. If the gap is any smaller, the risk of binding between the locking ring 39 and the capsule 25 may occur. As described below, the proximal end of the locking ring is also a radiopaque marker which guides the implanting physician as to the point where the prosthetic valve 1 is partially functional during deployment. The gap between the distal end of the locking ring 39 and the free edge of the polymer leaflet 111 is closely controlled to create a balance between optimal support and frictional engagement between the leaflet free edge 125 and the locking ring 39 as the over length of the valve 1 is shortened by expansion during deployment.

Referring to FIGS. 3A-3E, prosthetic valve support and transitional funnels are provided that are both engineered into the distal end of the catheter 19 and provided as separate kit components to provide a smooth transition when the prosthetic valve 1 is transitioned from the expanded configuration, the outside of the overall delivery system, to the collapsed or contracted configuration inside of the delivery system at the distal end of the catheter 19. The use of loading 35, support 37, and transitional 30 structure decreases the possibility of damaging a prosthetic valve 1 when inserted into the delivery system. The separate transitional for support funnel 37 may also be incorporated into the loading kit capsule supports 38a, 38b have upper and lower portion to give the distal end of the catheter 19 system greater column strength during loading. The capsule loading kit supports 38a, 38b are rigid plastic pieces that surround the distal end of the catheter 19 in a paired, annular fashion to establish a rigid region at the distal end of the delivery system and makes the distal most region of the catheter 19 substantially rigid during loading of the prosthetic valve 1. The loading kit supports 38a, 38b also maintain a protective structure concentric surrounding the delivery capsule and are removed during the TAVR procedure just prior to the fully loaded prosthetic valve 1 being inserted into the femoral artery using the delivery system as described herein.

FIG. 3A shows a loading kit assembly having a funnel-shaped portion 37 formed in loading kit supports 38a, 38b that engages the distal most portion of the catheter 19 to maintain structural integrity of the catheter 19 and the capsule 25 to facilitate a smooth transition of the prosthetic valve 1 from the partially expanded state to the collapsed configuration inside the catheter 19 during the valve loading procedure. Accordingly, funnel-shaped portions may be incorporated into or formed in any of the loading 35 or transitional 37 funnels as part of the loading kit or as a dedicated transitional funnel portion 30 permanently formed in the distal end of the catheter 19 or all of the above to provide for a sequential process of transitioning the prosthetic valve 1 from the expanded the collapsed or contracted configuration. Individual portions of the loading 35 or transitional 37 funnels may be designed having a sloped portion along all or a portion of a length along the axis of the catheter or a diameter of the catheter 19 to selectively form loading or transitional regions in any of the proximal end of the nosecone 10, the distal end of the catheter 19, the distal end of the locking ring 39 or as described with the dedicated funnel portion of FIG. 3B. The catheter may have a flat adjacent portion 31 arranged in an annular fashion surrounding the transitional funnel portion 30 and the guidewire lumen 14 for a closed mating engagement with a proximal end of the loading kit for engagement with a proximal end of the nosecone 10.

FIG. 3B is the combination of the nosecone 10, a separate distal loading funnel 35 containing a portion of the prosthetic valve 1. As shown in FIG. 3B, the distal loading funnel 35, the inner ring support member 20 and the distal portion of the catheter 19 traversed by the guidewire lumen 15 and the nosecone 10 and are separated in space to show the mating relationship of each prior to the valve loading procedure. The distal loading funnel 35 has a cuff 36 sized to accommodate the largest outer diameter of the prosthetic valve 1 in the expanded condition and assembled proximate to the end of the catheter 19 and as part of the loading kit assembly and maintains the prosthetic valve 1 in a partially compressed state such that the proximal and of the prosthetic valve 1 is assembled on the inner valve support 20 and shaped and manipulated to be introduced into the distal end of the catheter 19. An annular cuff 36 may contain a series of inner loading cones (not shown) that are that sequentially reduce the outer diameter of the prosthetic valve 1 during transition between the expanded configuration and the collapsed configuration for placement into the capsule 25 and the distal end of the catheter 19. For example, a first inner loading cone may reduce the outer diameter of the prosthetic valve from a first diameter to a second diameter or by a ratio between the first and second diameters, followed by a second inner loading cone reducing the outer diameter from the second diameter to a third diameter or by a ratio between the second and third diameters.

Accordingly, the distal loading funnel 35 may have inner and outer funnels spaced about the linear axis of the guidewire to facilitate reduction in the overall diameter of the prosthetic valve 1 that have a conical shape progressing from a larger inner diameter at the most distal funnel, that approximates the outer diameter of the prosthetic valve 1 in the expanded configuration and progresses to a most proximal portion of the most proximal loading funnel that contains an inner diameter that approximates the outer diameter of the prosthetic valve in the collapsed configuration. In this configuration, reduction of the maximum diameter of the prosthetic valve one is achieved both by introduction of the valve through the loading a funnel 35a together with passage of substantially the entire length of the valve through the funnel portion 35 and through the series of first and second inner loading funnels 35a, 35b as described in FIG. 3E. The inner and outer loading funnels 35a, 35b prevent valve leaflet and support structure infolding and asymmetric crimping by mechanically guiding both the outer diameter and inner diameter of the prosthetic valve 1 while it is being introduced into the distal end of the catheter 19 to be confined in the collapsed configuration. The distance between these two funnel surfaces as described below allows the prosthetic valve 1 to adopt the collapsed configuration while avoiding uneven folding of the leaflets the prosthetic valve 1.

FIG. 3C shows the next step in the loading procedure wherein the prosthetic valve 1 is drawn further into the loading funnel 35 such that the proximal portions 2 are disposed within the cavities 22 of the inner valve support 24 preparation with axial introduction of the inner valve support 20 into the distal portion of the catheter 19. FIG. 3D shows the distal loading funnel 35 in mating engagement with the distal end of the catheter 19 just prior to the loading process. When in close engagement, the minimum diameter of the dedicated transitional funnel 30 is roughly the same as the outer diameter of the compressed prosthetic valve 1. Thus, the minimum ID of the dedicated transitional funnel opening is equivalent and oriented line-to-line with the inner diameter of the capsule 25 so that the capsule does not experience shear force from insertion of the valve 1 into the catheter 19 during the loading procedure.

FIG. 3E shows an assembly of the distal portion of the catheter 19 comprising the capsule 25 and the progression of inner loading funnel and outer loading funnel 35a, 35b disposed on either side of an inner load cone locking ring 39. The series of loading funnels are a progression of cones having a progressively larger inner diameter of the most distal end to a progressively smaller inner diameter at the proximal and that approximates the dimension of the capsule number 25 in the prosthetic valve 1 in the collapsed or contracted state. The loading funnels may constitute additional members of an assembly kit provided in addition to the overall delivery device 60 and the prosthetic valve 1 wherein the loading funnels facilitate insertion of the prosthetic valve 1 into the distal most portion of the elongate catheter 39 while maintaining a preferred orientation for the individual leaflets as well as avoiding damage to the frame structure or valve leaflets of the polymeric valve 1 as the three dimensional design and orientation of the prosthetic valve 1 will exist when deployed at the native annulus compared to the compressed configuration necessary to insert the prosthetic valve 1 into the interior portion of the distal end of the elongate catheter 19.

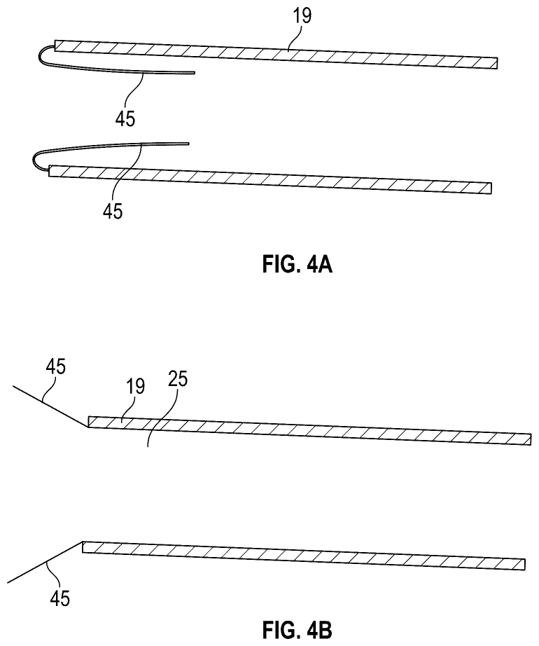

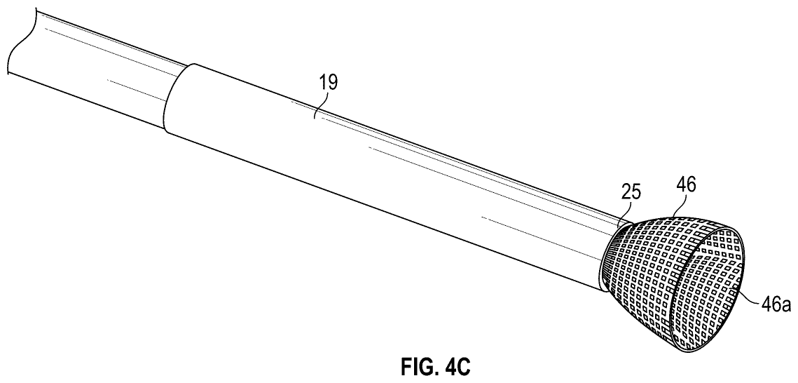

FIGS. 4A-4C are embodiments of a recapture system that deploys a tapered capsule or funnel distal of the terminal end of the catheter 19 using a recapture structure located internal to the outer surface of the distal portion of the catheter 19 but deployable therefrom as part of a recapture procedure. FIGS. 4A and 4B are one graphic concept of a recapture system having a deployable and rotatable pair of recapture extensions 45 that lie internal and substantially parallel to the capsule at the distal portion of the catheter 19 until deployed. The recapture extensions 45 deploy to form a dimension distal to the end of the catheter 19 that is larger than the diameter of the catheter 19. FIG. 4C shows a particular species of the recapture extensions 45 deployed into a tapered funnel shaped extension mesh 46 projecting from outer diameter of the capsule 25 in the distal end of the catheter 19 following deployment. Both FIGS. 4B and 4C show a configuration where the recapture extensions 45, 46 are deployed from within the capsule 25 to create a recapture funnel comprised of the recapture extensions 45, 46 extending beyond the most distal portion of the catheter 19 to create a distal opening adapted to receive the proximal end of the prosthetic valve 1, to draw the valve back into the catheter 19 if recapture after partial or complete deployment is required. In the alternate embodiment of FIG. 4C, the recapture means may be comprised of a sliding sleeve 46 having a diameter 46a that is extended from the distal end of the catheter 19 or is created by withdrawing the catheter 19 from around the sliding sleeve 46. The sliding sleeve 46 can be formed of a tapered conical, flared cone, trumpet shape formed from a shape memory metal that expands into a the recapture orientation cone having an opening shape with diameter 46a to receive the most proximal portion of the prosthetic valve 1 and tapered to the inner diameter of the capsule 25 as extended from the distal end of the catheter 19 to allow the prosthetic valve 1 to be drawn proximally through the opening 46a in the sliding sleeve 46 backward to recapture the prosthetic valve.

The embodiments of FIG. 4A-4C share the common principle of an inner recapture compressed within the outer diameter of the capsule 25 when maintained therein, but has a flared distal portion that is expandable, for example by retraction of an outer sheath member of the catheter to allow the partially or completely deployed prosthetic valve to be withdrawn into the native end of the catheter 19. FIGS. 4B and 4C shows a configuration where the recapture structure is formed as an annual or segmented inner layer inside the distal portion of the catheter 19 prior to being extended or expanded to receive the proximal portion of the prosthetic valve 1 to draw the valve back into the catheter if recapture after deployment is required. Additionally, the inner or outer diameter or both of the recapture feature may be coated with a biocompatible lubricant or has incorporated therein a layer or surface treatment that reduces the shear forces between the valve and the recapture feature upon drawing the prosthetic valve 1 back into the catheter 19 and may be established by a separate structure that would allow selective changes of the inner or outer diameter of the capsule 25 to allow selective increases or decreases in the inner or outer diameter thereof. A retaining suture or looped wire may be selectively engaged with a mating fixture on the prosthetic valve 1 to provide additional force to withdraw the prosthetic valve 1 back into the capsule 25.

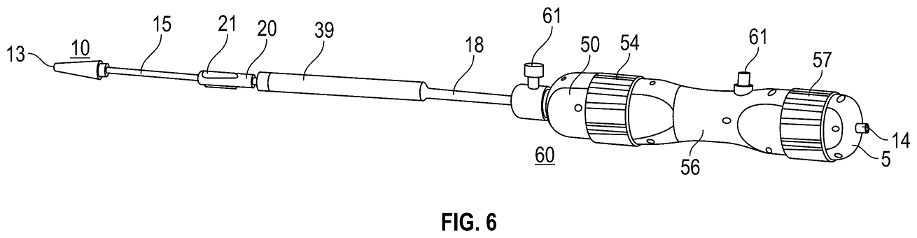

FIG. 5A-5D are a linear configuration of the elongated catheter element 19, 39 of the delivery system having a catheter body 19 formed from a flexible element 39 comprised of a series of laser cut spines 40 that enable the operator to flex the distal end 42 of the delivery system in a desired direction. The magnified section of FIGS. 5C and 5D show that the shape of the individual spines 45a, 45b, 45c . . . 45n (FIG. 5C); 46a, 456b, 46c . . . 46n (FIG. 5D) may take a variety of shapes comprising cut outs 45a, 45b, 45c or lateral extensions 46a, 46b, 46c from the body 47 of the flexible element 39 to form each individual element or cell of the spines 40 and are manufactured to facilitate the ability to change configuration along a linear axis of the flexible element 39 to allow the structure to be oriented by controls at the proximal end of the delivery system as described below at FIGS. 6-8.

Referring to FIG. 5B, a pull wire 152 internal to the flexible element 39 of the catheter 19 is retracted by the operator of the delivery system to manipulate the curvature of the distal portion 42 of the catheter 19 along the length of the flexible element 39. The capsule 25 is disposed within the distal portion 42 and is disposed offset to an intermediate section 41 that may be independently flexible at the junction between the intermediate section 41 and the flexible portion 39 and the distal portion 42 to alter the direction and relative orientation of the proximal portion of the catheter 19 and the distal portion 42 where the capsule 25 is maintained. FIG. 5B also shows the internal structure of the capsule 25 proximate of the nosecone 10 and surrounding the guidewire lumen 14. A portion of the capsule 25 is comprised of the laser cut hypo tube of the distal portion 42. The laser cut hypotube may be lined with Pebax at an outer diameter having an inner diameter lined with PTFE. A proximal portion of the capsule 25 comprises a locking ring 150 a flex layer hypotube 151 also having an outer diameter lined with Pebax and an inner diameter lined with PTFE. At the distal most portion of the capsule 25 an outer layer transitional ring 153 is circumferentially oriented around the most proximal portion of the capsule 25. FIG. 5E shows a configuration in which the rotational and flexible dimensions are predetermined and oriented in line with the anatomical orientation of the abdominal artery, descending thoracic aorta, aortic arch, and descending aorta to bring the prosthetic valve to deployment in the heart. The pull wire 152 is attached to the laser cut inner ring support member 154.

The laser-cut spines 40 along a portion or the entire elongate section of the catheter 19 provide both substantial linear and tensile strength along the bendable axis thereof during deployment. The orientation of the spines 40, collectively and in series, limits the plane in which the overall length of the flexible portion 39 of the catheter 19 may flex when incorporated into the complete delivery system under control by the surgeon. FIGS. 5C and 5D are magnified versions of the laser cut spines along the length of the catheter 19 showing how the orientation of the individual spines affects the flexibility characteristics along the length of the entire delivery system. As shown in FIG. 5E, the spin and flex direction are oriented in-line with the anatomical orientation of the ascending aorta, aortic arch and descending aorta to define a series of planes in which the delivery system can flex.

The predetermined curvature of the catheter 19 may also be controlled or dictated by proximal fixtures of the overall delivery system including the aforementioned pull wire, use of flush ports or lumens as aligning features, pull wire orientation and a pre-determined flex mechanism oriented by the laser cut of the spines. In one embodiment, the spines are laser cut from a hypo tube that has a predetermined configuration for the cut design that dictates the flexibility in different parts of the anatomy.

FIG. 6 is the overall configuration of the delivery system showing multiple individual components as described in the other Figures and as described herein showing the proximal handle portion manipulated by the surgeon, the elongated catheter and valve delivery structures located at the distal most portion of the delivery system. Viewing the overall system from the distal most atraumatic tip 13 of the nosecone 10, as described above, the guidewire lumen 15 traverses the inner valve support structure 20, the capsule contains the prosthetic valve (not shown) within and maintained in the compressed configuration the catheter 19 having the flexible element 39 that is advanced through the patient's vasculature following insertion such as through an incision proximate to the femoral artery. The handheld body 60 of the delivery system has a distal handle shell 50 and a deployment knob 54 that withdraws the capsule 25 at the distal end of the catheter to release the valve 1 from the collapsed configuration.

The deployment knob 54 is separated from a flex knob 57 by the middle handle shell 56. Rotation of the flex knob 57 reorient the position of the distal portion of the catheter 19 as described above in connection with FIG. 5. The deployment knob 54 and the flex knob 57 both rotate in a circumferential fashion and, because both are disposed within the body of the handheld body 60, can readily be manipulated independently or simultaneously by the surgeon to both orient the prosthetic valve 1 relative to the native valve annulus and to deploy the valve 1 from the capsule 25. The reorientation is achieved by exerting tension on an internal pull wire 152 that traverses the entire length of the catheter 19 and is typically embedded in the interior polymer coating thereof. Proximate to the flex knob 57 is a proximal handle shell 59 that surrounds the guidewire lumen 15 that runs from the most proximal portion of the handheld body 60 through to the terminal end embedded in the nosecone 10 as described in connection with FIG. 1B

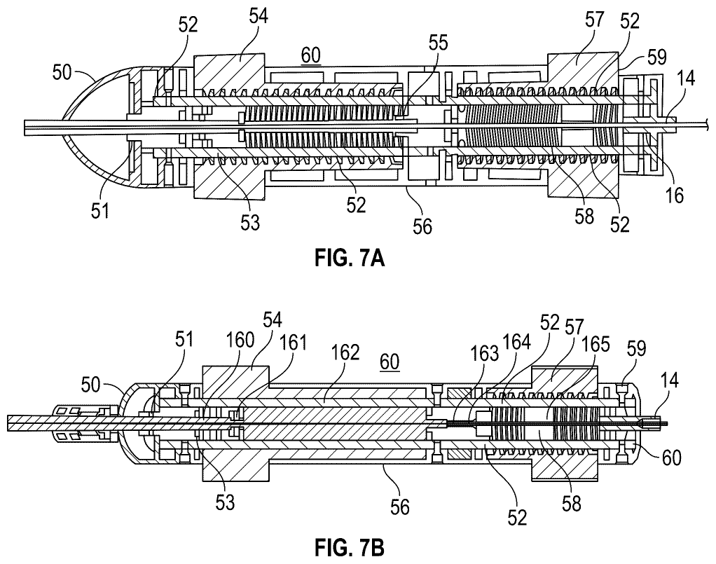

FIG. 7A is an internal view of the handheld body containing a rail mounted design of the delivery system operably connected to the deployment knob 54 in the flex knob 57 to provide both flexible steering and deployment knobs accessible around the outer surface handheld body 60 that are manipulated by the surgeon during the TAVR procedure. The rail mounted delivery system design combines and holds together the external structural components outside the individual body handle 60 components as well as the internal mechanical features that enable 360 degree accessibility to deployment knob 54 and steering deployment knob 57 thereby allowing the physician easier access to the control knobs and enhancing the speed, ease and smoothness of valve delivery in a TAVR procedure.

Continuing to refer to FIG. 7A, a distal handle shell 50 is a structural component at the distal and of the handheld body 60 that circumferentially surrounds the stationary layer handle adapter 51 and is traversed by the guidewire 15. As described below, the stationary layer 62 is traversed by the capsule 25, inner ring and guidewire lumen 14. The stationary layer handle adapter 51 is fixed in place relative to the exterior of the handheld body 60 and the distal handle shell 50, mid handle shell 56 and proximal handle shell 59. A pair of mounting rails 52 run in a substantially parallel configuration through the interior of the handheld body 60. The capsule handle adapter 53 surrounds the proximal portion of the capsule such that rotation of the deployment knob 52 retracts the capsule 25 proximally to deploy the prosthetic valve 1 from the distal end of the catheter 19. The inner ring handle adapter 55 is disposed between the deployment knob 54 and the flex knob 57. A pull wire handle adapter 58 is operably engaged with the flex knob 57 such that circumferential rotation of the knob withdraws the pull wire 152 proximally to flex and orient the distal end of the catheter 19 as described above. The proximal handle shell 59 contains a guidewire lumen handle adapter 60 that maintains the guidewire lumen 14 centrally disposed in a linear axis that traverses the entire length of the handle body 60. The guidewire lumen allows proximal access for insertion of the guidewire 15. A C-shaped marker band is disposed in the capsule 25 and aligned with the direction of flex direction along the catheter 19.

FIG. 7B is an internal view of the delivery system handle 60 showing the orientation of the outer layer hypotube 160 just distal of the outer layer termination lead screw 161. The outer layer barrel 162 is disposed intermediate of the handle body 60. At the proximal end, the flex layer hypotube 163 terminates at the flex layer termination block 164. The pull wire 152 and the pull wire lead screw 165 are shown in engagement with the flex knob 57 and the flex layer barrel 166.

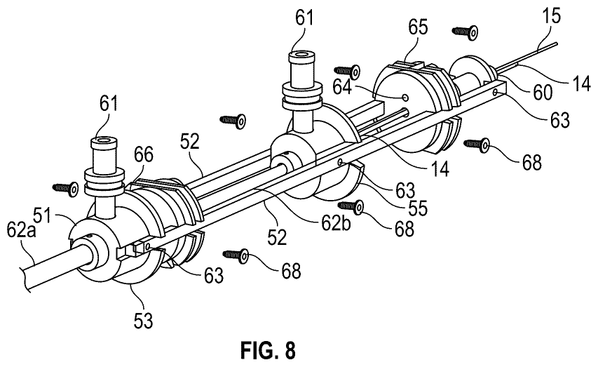

FIG. 8 is an internal view of the rail based design of the interior of the handheld body 60. A stationary layer 62a has an axial length that runs substantially the entire axial distance of the catheter 19 and has a proximal portion fixedly attached and embedded in the stationary layer handle adapter 51. The stationary layer 62a extends along most of the length of the delivery system and ends at a point where if the in-line sheath was fully advanced, the sheath would rest on the stationary layer to avoid removing the stationary layer 62a from the transfemoral access site as the capsule 25 is pulled back when the prosthetic valve 1 is deployed. The stationary layer 62a stabilizes the delivery system during the deployment procedure.

The capsule handle adapter 53 circumferentially surrounds a portion of the stationary layer 62a containing the layer. The mounting rail 52 is attached to the outer handle pieces by rail attachment screw 68 that engage the rail attachment holes 63. Rotation of the deployment knob 54 causes proximal linear movement of the capsule handle adapter 53 along the length of the mounting rails 52. As shown in FIGS. 7-8, the capsule handle adapter 53 is circumferentially surrounded by the deployment knob that is rotatable 360° from the exterior of the handheld body 60 the superior portion of the capsule handle adapter 53 has a groove 56 that facilitates engagement with the distal handle shell 50 and mid handle shell 56 and maintains the capsule handle adapter 53 in a fixed orientation relative to the outer body during linear movement of the capsule handle adapter 53. An inner lumen 62b traverses the capsule handle adapter 53 and engages and is embedded in the inner ring handle adapter 55. The inner lumen 62b is disposed circumferentially within the stationary layer 62a and is slidable along the length thereof. Both the capsule handle adapter 53 and the inner ring handle adapter 55 have one-way valves extending from the interior of each structure through the shell of the handheld body 62 permit venting of the internal space of both the stationary layer 62a and the inner lumen 62b to ambient air.

Continuing to refer to FIG. 8, the proximal portion of the handheld body 60 has a flex handle adapter 65 that slides along the mounting rails 52. The proximal handle adapter has an aperture 64 for receiving and securing the flex pull wire (not shown). As shown in FIG. 7, a guidewire lumen handle adapter 60 is circumferentially oriented around the guidewire loop lumen 14 and the guidewire 15. The capsule layer is inside the stationary layer and the capsule handle adapter 53 which slides forward and back along the rails 52. The capsule layer is not rigidly fixed to the rails 52. Within the handle 60, the capsule handle adapter 53 and the flex handle adapter is attached to the pullwire 152 to exert tension on the locking ring 150.

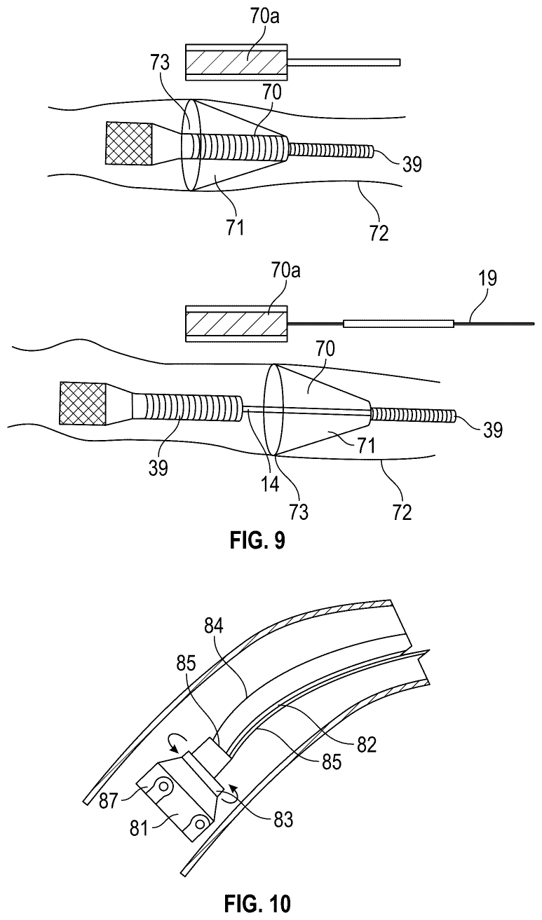

FIG. 9 is an embolic protection mechanism 70 that may be oriented and deployable in an annular configuration around the guidewire lumen 15 that can be deployed during a TAVR procedure between the prosthetic valve 1 and the downstream blood flow of the vasculature 72 of the patient at a point along the catheter 19. Particularly during the deployment of the prosthetic valve 1 at the native annulus, and during introduction and advancement of the catheter 19 along the vasculature 72 from the femoral artery to the heart, a risk of generating emboli results from either the motion of the catheter 19 through the vasculature 72 (see FIG. 5B) or from placement of the prosthetic valve 1 at the native annulus (not shown). Once deployed, the embolic protection device 70 surrounds the catheter 19 and has a shape that closely engages with the side wall of the vessel 72 to cause substantially all of the blood flow to pass therethrough. In one embodiment, the embolic protection device 70 is formed in a funnel or umbrella form 71 that has an outer surface 73 to engage the sidewalls of the vessel 72 and acts as an in-line filter to allow the passage of small red blood cells and other serum proteins catching larger emboli, such as mineral structures and tissue fragments. The embolic protection device 70 may be located proximate to the portion of the catheter 19 containing the capsule 25 and the prosthetic valve 1 or may be located proximally along the length of the catheter 19.

In another embodiment, the embolic protection device 70a may be deployed separately and introduced along the length of the catheter 19 and subsequently deployed in an annular fashion around the catheter 19. The funnel or umbrella portion 71 of the embolic protection device 70 may be formed of a shape memory metal, such as a Nickel Titanium wire or mesh that is activated by a wire, outer lumen, (not shown) or other mechanism associated with the catheter 19 or through heat expansion upon exposure to the bloodstream. The mesh structure of the funnel portion 71 may be formed directly from the shape memory properties of the material, or may be coded with an inner layer of a biocompatible material having porous openings designed to permit the free flow of red blood cells, white blood cells and other serum components, such as plasma proteins, while retaining larger embolic particles formed from calcium, or other solid structures, or tissue fragments. In either case, the embolic protection device is either withdrawn entirely or collapsed onto the outer diameter of the catheter 19 prior to withdrawing the entirety of the delivery system from the vasculature of the patient. The overall dimension of the embolic protection device 70 has an outer dimension at the outer edge 73 that is positioned along the length of the catheter 19 allowing deployment along the ascending aorta and proximal of the deployed prosthetic valve 1 but below the brachiocephalic artery, left carotid artery, and left subclavian to avoid entry of debris into these vessels.

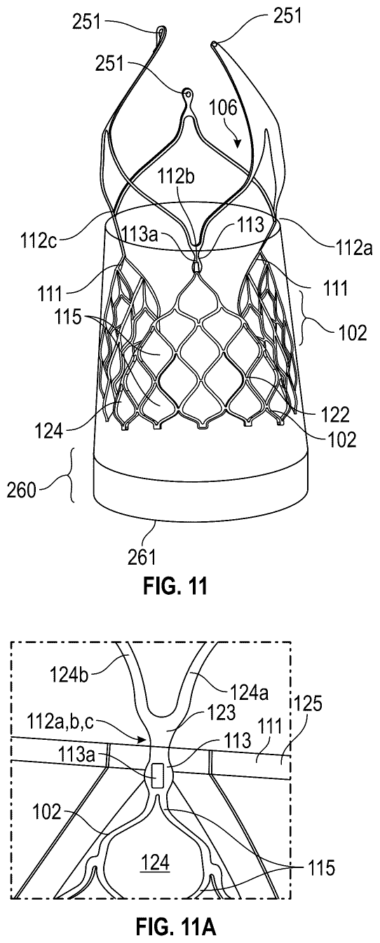

FIG. 10 is a delivery system assembly 80 provided at the distal most portion of the catheter 19 and has a rotatable valve capsule 81, internal of a locked distal housing 87, disposed therein. The assembly 80 is comprised of an outer shaft 84, and inner shaft 85 that contains the prosthetic valve 1 in a collapsed configuration and having a length that terminates within the locked distal housing 87 such that the inner rotatable valve capsule 81 can the selectively rotated relative to the locked distal housing 87, preferably with a fixed orientation of the rotatable valve capsule 81 to the commissures of the prosthetic valve 1 contained at the distal end of the catheter 19 and contained within the rotatable valve capsule 81. Manipulation of a control knob or fixture in the handheld body 60 of the delivery system assembly 80, for example by manipulation of a fixture that controls pull wire 82, allows the surgeon to selectively and rotatably position the prosthetic valve 1 structures within the rotatable valve capsule 81 independently of establishing a selected length of the catheter 19 relative to and within the native annulus.

In use, the prosthetic valve 1 would be located and oriented at the native annulus by the surgeon who selectively positions the prosthetic valve 1 on a rotational axis prior to deployment, particularly taking into account support structures for the prosthetic valve 1, commissures of the prosthetic valve, relative to existing native structures prior to deployment, such as to accommodate features of individual patient anatomy or to avoid coronary arteries proximal to placement of the prosthetic valve 1. The inner and outer shafts 85, 84 rotate together or independently to orient the valve within and relative to the rotatable internal capsule 81.

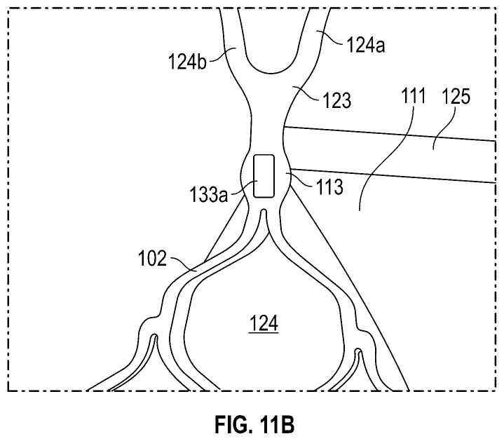

FIG. 11 is a perspective view depicting an example of a prosthetic valve 1 for use with the present delivery system invention. The prosthetic valve 1 has a frame 102 that acts as a support structure for the implantable prosthetic valve body 1. As noted above, this embodiment can be reduced in size from an expanded to a contracted state. In the embodiment of FIG. 11, the body of frame 102 includes multiple struts 122 coupled together in a unitary or monolithic body. Each strut 120 is coupled with another strut, e.g., at a location 122, that is deformable for transition of frame 102 between the expanded and collapsed or contracted states (FIG. 11 shows the frame 102 in an expanded state). In this embodiment, struts 122 are interconnected in a crossing pattern, or lattice, such that multiple open regions 124 are present. These open regions 124 generally have a four-sided rounded diamond shape in the expanded configuration shown in FIG. 11. The open region 124 and the struts 120 forming the sides (four or more struts in this embodiment) of open region 124 are together referred to as a cell 115 of the frame. Many of the struts 120 are generally oriented at an angle with respect to longitudinal axis 110.

At the downstream 108 side of frame 102, there are three commissure positions 112a, 112b, 112c. Each commissure position aligns with the intersection between two adjacent leaflets 111 and is shown in a partial open state in FIG. 11. This particular embodiment is configured for use with a three leaflet valvular body, but the analogous design can be used for a valve with fewer or more leaflets and the total interior and outer diameter as well as the axial length along an axis 110 can be altered without changing the proportions and dimensions disclosed herein.

At the upper portion of this position 112, a commissure stabilization element 113 is formed as a rivet disposed near the commissure and proximate to the joinder region of two adjacent leaflets 111 therebetween and is oriented proximate to a cell 115. Rivet 1130 also recruits additional polymer material surrounding the rivet 113 itself as well as the surrounding portion of the frame 102 also establishes a deflection attenuation feature that advantageously stiffens that portion of frame 102. In addition to providing the commissure stabilization function the rivet feature 113 attenuates excess deflection of the leaflets 111 and the overall frame 101 under full diastolic loading. In this example, the rivet feature 113 is oval-shaped having an aperture 113a that accumulates and recruits additional polymer surrounding and traversing the rivet feature 113 and that is disposed in a central portion thereof, although in other embodiments the feature may have an alternative arrangement that satisfies the function of recruiting additional polymer and providing the structural and stabilization functions proximate to the commissure as described herein.

Referring again to FIG. 11, along the edge of the frame 102, towards the downstream 108 end, and displaced from the frame of the prosthetic valve 1 and towards the downstream and 108, a portion of the frame connected to the rivet feature 113 projects in the direction towards the downstream and 108 and is formed as a continuous portion connected with the frame 101 and is generally aligned with the struts 122 that are aligned with the commissure portions 112a,b,c and beyond the upstream edge of the leaflets 111. This configuration provides both structural support to the stent frame 101, structural support to the leaflets 111 and commissures 112a,b,c and minimizes leaflet stress across the attachment point to the commissures and to the leaflets 111 proximate to the attachment points. Each cell 115 of the frame 102 can be uniquely designed and oriented to align the geometry of the individual cells 114 to reach a peak or apex at the commissures 112a,b,c in a generally triangular shape having an apex coinciding with the rivet feature 113.

The commissure stabilization function of the rivet feature 130 is shown in greater detail in FIGS. 11A and 11B. In the downstream direction 108, the frame 101 assumes a unique configuration surrounding the rivet feature 113. In the upstream direction, the strut feature is integral with cells 115, the frame 101 and supporting struts 121. Any downstream direction, the rivet feature 113 is integral with a superior strut region 123 that is a single, linear portion of the frame 101 disposed axially in the downstream direction 108. A portion of the superior strut structure 123 is co-linear along a horizontal axis of the upper leaflet edge 125 and is surrounded by a common polymer layer at the commissure 112 to provide additional support as described above. A portion of the superior strut structure 123 is also disposed in a downstream direction 108, relative to the upper portion of the upper edge 125. As shown in FIG. 11B, the upper leaflet edge 125 as well as a portion of the leaflet 111 may share a common horizontal axis with the aperture 113a of the rivet feature 113. As noted above, at any point along the upper portion of the frame 102 proximate to the rivet feature 113, surrounding the rivet feature 113 itself, including aperture 113a, proximate to and surrounding the superior strut structure 123, a thicker depth and volume of the polymer used to form the leaflet accumulates around any or all of the foregoing structures.

Referring again to FIG. 11, the downstream end 108 of frame 120 of prosthetic valve 1 includes a halo 250 located upstream to commissure positions 112. In these embodiments, halo 250 is comprised of a single structure or wire that extends from each commissure to create a superior halo frame 124a, 124b and terminating in a series of halo apices 251a,b,c that are equivalent in number to the number of commissures 112 and generally disposed intermediate to the commissures such that the overall length of each superior frame is approximately equal and the orientation of the series of crown apices is disposed in a spatial orientation that is roughly equidistant from each commissure along a linear axis thereof perpendicular to linear axis 110 and traversing the series of cells 115 that comprise the commissure support portion of the frame 102 that is integral with the frame 102 and passing through the body frame at the rivet feature 113 and through the superior strut 123. The orientation of the halo apices 151 includes a location which is both downstream of the commissures and outside of any alignment of a linear access of the commissures that is coincident with axis 110.

The superior frame portions 124a, 124b form an hourglass configuration upon a length thereof between the commissure 112 and the apices 151 to create a flare along the length thereof that establishes an inner and outer diameter, at the most exterior outer diameter portion thereof, that is approximately equal to the outer diameter of the frame 102 such that diameter D1 has the same outer diameter as diameter D2 as shown in FIG. 11. Although the embodiment of FIG. 11 shows a single wire between the commissures 112 and the halo apices 250, the structure of the superior frames 124a, 124b may comprise additional structural portions along a length thereof that are integral with the superior frame portions 124a, 124b.

Halo 250 can aid in reducing deflection experienced by the frame 102 that can lead to lower valvular leaflet stresses. Additionally, crown 250 can function as a straightening feature to help stabilize inflow side 106 if incorrectly implanted at an oblique angle. If the native annulus is oval, halo 250 can also help keep the prosthetic valve 1 and a circumferential orientation when placed in the native annulus therefore functioning with lower stress. Crown 2500 can also provide additional anchoring in the sinutubular junction (STJ). The embodiment of FIG. 11 A-B expressly include an embodiment where the halo 250 is free of polymer and may assume a configuration upon loading of the prosthetic valve into the catheter 19 such that the portions of the halo 250 are manipulated to engage the mirror image cavities of the inner valve support feature (see FIGS. 2A-2B) when aligned upon transition of the prosthetic valve 1 from the expanded to the collapsed or contracted state as shown in the series of FIG. 2 and FIG. 3. the valve straight and/or aligned if deployed at an angle. A material layer or coating including biocompatible materials that may comprise a polymer or therapeutic substance such as an anticoagulant, or anti-thrombotic, may be placed on the exterior of the halo along a length or the entirety thereof and especially at the strut portion. The halo 250 may also allow for the release of the prosthetic valve 1 at a later stage in the deployment process by retaining a portion of the halo 250 and engagement with the distal portion of the catheter 19 as described in the recapture embodiment of FIG. 4 and accompanying text. In practice, the retention of any portion of the halo portion 250 within or connected to the catheter 19 allows the surgeon to assess anchoring of the prosthetic valve 1 and/or flow parameters through the native annulus without fully releasing the prosthetic valve 1 from the delivery system.

The apices 251 of the halo can be comprised of a terminal locking feature in the form of a loop or eyelet that is disposed and oriented in the same manner as the individual apex 251 to form an extension of the structure of the apices 250 that is generally internal of the flared diameter D1 of the halo 250 and integrally formed with the superior strut 123 and the superior frame portions 124a, 124b.

Referring again to FIG. 11, prosthetic valve 1 is comprised of a sealing skirt 260 having a sealing skirt edge 261 at the inflow portion 106 of the prosthetic valve 1. In various embodiments of the prosthetic valve 1, the formation of the frame 101, the leaflets 111 can include forming inner and outer polymer layers associated with the frame 101 and either or both of a sealing skirt that covers the sealing skirt edge 261 that may include forming a separate inner liner (not shown). The sealing skirt 160 may be formed as disclosed below and may include additional steps of finishing (e.g., trimming) any formed polymeric structure performed at the desired point in the manufacturing methods of the prosthetic valve 1.

In a preferred embodiment for manufacturing the prosthetic valve 1, the sealing skirt 260 is formed by electric field assisted spray deposition (e.g., electro-spinning) to form the structural portion of the valve encompassing the entirety of the inflow portion 106 and encompassing the sealing skirt edge 261 component of the prosthetic valve 1. The electrospun forming the sealing skirt 260 polymer can be formed directly onto the structure of the prosthetic valve 1 during the manufacturing process when the frame 102 is either of a bare metallic frame or a polymer coated frame 102 or can be electrospun onto a former mold, mandrel or other substrate and then separately attached to a leaflet support structure.

An electrospun component of the prosthetic valve 1 can comprise any or all of desired polymer component of the valve, such as the sealing skirt, an inner liner, an outer liner, or a sewing cuff (for surgically implanted valves). The below description applies to electrospinning of a polymer onto the metal frame structure 101 but could also be applicable to a prosthetic valve comprising polymer frame-like structure that forms a portion of the frame 101 or overall support structure of the prosthetic valve 1. The use of an electrospun coating has shown strong bonding properties necessary for the attachment of the electrospun portion to either of a polymeric structure or a metallic frame. This separate application of an electrospun layer has a number of advantages over traditional dip-casting or molding of polymer for valves such as, for example, improved adhesion of the electrospun polymer to a metallic frame, excellent adhesion of the electrospun polymer to a dipped or cast polymer, and excellent adhesion of permeable electrospun polymer to impermeable electrospun polymer.

Electrospinning involves utilizing a fiber from a polymer substrate to form a desired structure. Electrospinning can use a high electrical current to draw a charged solution of polymer onto a charged collector and can create structures having features on the micron and even nanometer scale. By varying the production parameters, solution conditions, and collector design, various morphologies can be constructed and can be tailored to the desired application. The morphology of the electrospun structure can include, for example, a lattice construct of fibers that can be conformable to abnormal geometries and spikes of calcium typically seen in stenotic heart valves.

These electrospun polymer structures can be formed using any type of polymer, including all SiPUU or other polymers described herein, and the type of polymer used for one structure (e.g., the leaflets) can be the same or different from the polymer used to form another structure (e.g., sealing skirt or sewing cuff). Multiple different materials can be used to form each structure as well. The electro-spinning process can incorporate heterologous materials to form a blended or copolymer that can incorporate separate nonreactive structures into the electro-spun structure or can incorporate therapeutically active compounds such as heparin or other anticoagulants, conjugated antibodies to the polymer surface, and other therapeutic compounds commonly used with drug-eluting stents, tissue-based prosthetic valves, and vascular grafts and stents.

The embodiments below are described with respect to dry-spinning (with use of a whipping jet) and wet-spinning (without use of a whipping jet) types of electrospinning, which are familiar to those of ordinary skill in the art. These techniques can be used to electrospin polymers with varying degrees of fluid (e.g., blood) permeability, from impermeable or substantially impermeable polymers to polymers of relatively low or relatively high permeability.

Referring again to FIG. 11, the electrospun sealing skirt may be attached to the prosthetic valve 1 by a dry-spinning process. The electrospun sealing skirt includes a construct of polymer fibers that form lattice or web to provide the circumferential skirt 260 and the sealing skirt edge 261 surrounding the inflow portion 106 of the prosthetic valve 101. The construct of the individual polymer fibers is fabricated from a SiPUU and extends across or bridges the gap within each frame cell 115 and the open spaces 124 disposed therebetween. This highly porous, conformable polymer material is crimpable, seals irregularities at the annulus, and encourages healthy in-growth of tissue for long term anchoring. This dry-spinning process can also be combined with a wet-spinning process (or the wet spinning process can be used alone) to create an impermeable layer on the inner diameter (ID) of the prosthetic valve 1 to encourage laminar flow on the inner lumen of the frame, while also creating a robust sealing layer on the outer surface that is in contact with the native annulus. In the embodiment of FIG. 11, the sealing skirt 260 has a uniform height spanning the linear dimension the cost axis 110 from the sealing skirt edge 161 toward the outflow portion of the valve 108 and a generally uniform thickness and the annular dimension as the sealing skirt 261 is applied to the circumferential interior and/or exterior of the inflow portion 106 of the prosthetic valve 1, although either of the of the thickness or the height thereof can be selectively altered at any point of either the depth or height dimension if desired.

As used herein and in the appended claims, the singular forms “a”, “an”, and “the” include plural referents unless the context clearly dictates otherwise.

However, in no way should this specification be interpreted as implying that the subject matter disclosed herein is limited to a particular value or range of values absent explicit recitation of that value or range of values in the claims. Values and ranges of values are provided herein merely as examples.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

All references cited herein, including but not limited to patents and patent applications, are incorporated by reference in their entirety.

What is claimed is:

1. A delivery system to orient and deploy a prosthetic valve at a native annulus comprising:
an elongate catheter having a cavity flexible portion along the length thereof and terminating in a distal nosecone;
an inner valve support circumferentially disposed around the elongate catheter and located proximally to the distal nosecone, wherein the inner valve support is shaped to accommodate the most proximal portion of the prosthetic valve in a compressed configuration;
a capsule containing the prosthetic valve in the compressed configuration;
a handheld body containing a steering control to orient the shape of the distal end of the elongate catheter and a deployment control to release the prosthetic valve.

2. The delivery system of claim 1, further comprising the prosthetic valve having a proximal portion shaped to be maintained in conforming engagement with the inner valve support when the prosthetic valve is in the compressed configuration.

3. The delivery system of claim 1, wherein the inner valve support is formed of a plurality of channels that contain the proximal portion of the prosthetic valve.

4. The delivery system of claim 3, wherein the channels are ahped as a mirror image of the proximal portion of the prosthetic valve.

5. The delivery system of claim 1, wherein the elongate catheter is comprised of a guidewire that terminates at a point proximal to the distal end of the nose cone and within a body of the nosecone.

6. The delivery system of claim 1, wherein the nose cone has an annular proximal portion circumferentially surrounding the guidewire and shaped to engage an inner portion of the most distal portion of the prosthetic valve.

7. The delivery system of claim 1, wherein the distal end of the elongate catheter has a tapered surface between an outer diameter of the elongate catheter and in inner diameter of an interior space of the catheter containing the capsule.

8. The delivery system of claim 1, in combination with the prosthetic valve and a distal loading funnel having a cuff sized to accommodate the largest outer diameter of the prosthetic valve in an expanded condition.

9. The delivery system of claim 8, further comprising loading kit capsule supports surrounding the surrounding the distal end of the elongate catheter in a paired, annular fashion to establish a rigid region at the distal end of the elongate catheter.

10. The delivery system of claim 1, wherein the flexible portion of the elongate catheter is comprised of a laser cut spine.

11. The delivery system of claim 1, wherein the flexible portion of the elongate catheter has a predetermined curvature.

12. The delivery system of claim 1, wherein the steering control for the distal end of the elongate catheter is a flex knob that rotates in circumferential fashion around the handheld body.

13. The delivery system of claim 1, wherein the deployment control for the prosthetic valve is a deployment knob that rotates in circumferential fashion around the handheld body.

14. The delivery system of claim 1, wherein the handheld body contains a pair of mounting rails oriented in substantially parallel continuation through the interior of the handheld body and operatively engaged with the steering control and the deployment control mechanisms.

15. The delivery system of claim 1, further comprising a stationary layer that is traversed by the guidewire, the capsule, and is fixed in place relative to the exterior of the handheld body.

16. The delivery system of claim 1, further comprising a locking ring fixedly attached to an interior of the handheld body.

17. The delivery system of claim number 1, further comprising a recapture mechanism deployable from the distal end of the elongate catheter and having an outer diameter greater than the outer diameter of the prosthetic valve.

18. The delivery system of claim 1, further comprising a deployable embolic protection mechanism surrounding the elongate catheter and having a diameter to closely engage with a sidewall of a native vessel to cause a substantial portion of blood flow to pass through the embolic protection mechanism when deployed during placement of the prosthetic valve at the native annulus.

19. The delivery system of claim 1, wherein the elongate catheter is further comprised of a rotatable capsule having a rotational axis around the elongate catheter to selectively and rotatably position the prosthetic valve at the most distal end of the delivery system.

20. The delivery system of claim 1, further comprising the prosthetic valve having a frame portion comprising a plurality of cells comprising interconnected struts that are deformable for transition of a frame of the prosthetic valve from the collapsed to the expanded configuration.

21. The delivery system of claim 20, wherein the frame is comprised of commissure support positions aligned at an intersection between adjacent valve leaflets.

22. The delivery system of claim 21, wherein the valve leaflets are formed from a polymer.

23. The delivery system of claim 20, wherein the frame has a downstream and comprising a halo located upstream to the commissure support positions.

24. The delivery system of claim 23, wherein the halo is shaped to be maintained in conforming engagement with the inner valve support when the prosthetic valve is in the compressed configuration.
